(19) United States
(12) Patent Application Publication
(10) Pub. No.: US 2021/0087271 A1
(43) Pub. Date: Mar. 25, 2021

(54) FC VARIANTS WITH REDUCED EFFECTOR FUNCTION

(71) Applicant: Adimab, LLC, Lebanon, NH (US)

(72) Inventors: Robert Pejchal, Lebanon, NH (US); Eric Krauland, Lebanon, NH (US); Maximiliano Vasquez, Lebanon, NH (US); Michael Brown, Lebanon, NH (US); Anthony Cooper, Lebanon, NH (US)

(21) Appl. No.: 17/024,749

(22) Filed: Sep. 18, 2020

Related U.S. Application Data
(60) Provisional application No. 62/903,164, filed on Sep. 20, 2019.

(51) Int. Cl. C07K 16/28

(52) U.S. Cl. CPC C07K 16/2809; C07K 2317/31; C07K 2317/21; C07K 2317/24; C07K 2317/734; C07K 2317/524; C07K 2317/71; C07K 2317/92; C07K 2317/732; C07K 2317/53

(57) ABSTRACT

The present invention provides Fc variants and polypeptides, e.g., antibodies and Fc fusion proteins, comprising such Fc variants. In particular, Fc variants with diminished effector function as a consequence of hinge region and CH2 domain mutations, e.g., LALE-PG, are provided. Such variants maintain antigen-binding and favorable developability profiles and may display improved expressability.

### RELATED APPLICATIONS

This application claims priority to U.S. Provisional Application No. 62/903,164, filed on Sep. 20, 2019, the contents of which are incorporated by reference in their entirety.

### FIELD OF THE INVENTION

The invention relates to engineered polypeptides, e.g. antibodies, comprising Fc variants with amino acid substitutions in the hinge region (L234, L235) and CH2 domain (P329) resulting in diminished effector function.

### BACKGROUND OF THE INVENTION

Biologics, such as antibodies and antibody-based molecules, represent attractive candidates as diagnostic tools and therapeutics (Reichert, mAbs, Vol. 5(1), pp. 1-4 (2013)). To date, more than 70 therapeutic monoclonal antibodies have been approved for and successfully applied in diverse indication areas including cancer, organ transplantation, autoimmune and inflammatory disorders, infectious disease, and cardiovascular disease.

The Fc region of an antibody, i.e., the C-terminal portion of the heavy chains of an antibody that spans domains CH2, CH3 and a portion of the hinge region, is involved in effecting the physiological roles played by the antibody. The effector function attributable to the Fc region of an antibody varies with the class and subclass of antibody and includes binding of the antibody via the Fc region to a specific Fc receptor (“FcR”) on a cell which triggers various biological responses. FcRs are expressed in a variety of immune cells such as monocytes, macrophages, neutrophils, dendritic cells, eosinophils, mast cells, platelets, B cells, large granular lymphocytes, Langerhans' cells, natural killer (NK) cells, and T cells. Upon Fc binding to FcRs, effector cells are recruited, resulting in subsequent immune responses such as release of inflammation mediators, B cell activation, endocytosis, phagocytosis, and cytotoxic attack. The ability of an antibody to mediate cytotoxic and phagocytic effector functions is a potential mechanism by which the antibody destroys targeted cells.

The receptors for the Fc region of IgGs are a family of transmembrane glycoproteins comprising three different receptor types having different binding specificities:FcγRI, FcγRII, and FcγRIII (Hulett and Hogarth (Hulett, M. D. and Hogarth, P. M., Adv. Immunol. 57 (1994) 1-127)). Engagement of the Fc region of IgGs with these FcγRs governs antibody dependent cell-mediated cytotoxicity (ADCC) and antibody dependent cell-mediated phagocytosis (ADCP). In humans, the FcγR protein family includes FcγRI (CD64); FcγRII (CD32), including isoforms FcγRIIA, FcγRIIB, and FcγRIIC; and FcγRIII (CD16), including isoforms FcγRIIIA and FcγRIIIB (Raghavan and Bjorkman, Annu. Rev. Cell Dev. Biol. 12 (1996) 181-220; Abes, et al., Expert Reviews (2009) 735-747). Some FcγRs are activating receptors, e.g., FcγRI, FcγRIIA/C, and FcγRIIIA, characterized by an intracellular immunoreceptor tyrosine-based activation motif (ITAM), whereas other FcγRs are inhibitory receptors, e.g., FcγRIIB, containing an inhibitory motif (ITIM). Likewise, the affinity of the FcγRs also vary: FcγRI binds monomeric IgG with high affinity, whereas FcγRIII and FcγRII are low-affinity receptors, interacting with complexed or aggregated IgG. Another type of IgG Fc receptor is the neonatal Fc receptor (FcRn). FcRn is structurally similar to major histocompatibility complex (MHC) and consists of an α-chain noncovalently bound to β2-microglobulin.

In certain situations, it can be advantageous to decrease or eliminate antibody effector function, e.g., an antibody is intended to engage cell surface receptors and prevent receptor-ligand interactions (antagonists) and/or preventing antibody-drug conjugates from interacting with FcγRs leading to off-target cytotoxicity. IgG binding to FcγRs depends on residues located in the hinge region positions 233-239 (EU numbering) and the CH2 domain. IgG binding to C1q has also been reported to involve CH2 domain residues, e.g., positions 318, 320, 322, and 331 (Duncan and Winter (Nature 332:738-40 (1988); Tao et al., J. Exp. Med., 178:661-667 (1993); Brekke et al., Eur. J Immunol., 24:2542-47 (1994)) and lower hinge region residues (Alegre et al., J Immunol. 148:3461-3468 (1992); Xu et al., Cell Immunol. 200:16-26 (2000); WO1994/29351). A number of IgG Fc region variants comprising amino acid substitutions in these regions are known in the art (see e.g., Oganesyan, et al., Acta Cristallographica D64 (2008) 700-704, disclosing L234F/L235E/P331S; WO2012/130831 (Roche), disclosing L234A/L235A/P329G; WO2015/077891 (Zymeworks), providing a review of other known modifications to reduce FcγR or complement binding to the Fc in Table C (e.g., GSK-N297A; Ortho Biotech—L234A/L235A; Protein Design Labs—IgG2 V234A/G237A; Wellcome Labs—IgG4 L235A/G237A/E318A; GSK—IgG4 S228P/L236E; Alexion—IgG2/IgG4combo; Merck—IgG2 H268QN309L/A330S/A331S; Bristol-Myers—C220S/C226S/C229S/P238S; Seattle Genetics—C226S/C229S/E323P/L235V/L235A; Medimmune—L234F/L235E/P331S; Trubion—Hinge mutant, possibly C226S/P230S).

Substitution of human IgG1 and IgG2 residues at positions 233-236 and IgG4 residues at positions 327, 330 and 331 has been shown to significantly reduce ADCC and CDC by disrupting FcγR and C1q binding, respectively (see, e.g., Armour, et al., Eur. J. Immunol. 29(8) (1999) 2613-2624 and Shields, et al., J. Biol. Chem. 276(9) (2001) 6591-6604). For example, the triple mutation L234F/L235E/P331S has been shown to decrease binding activity to C1q, FcγRI, FcγRII and FcγRIIIA (Oganesyan, et al., Acta Cristallographica D64 (2008) 700-704). Substitutions at positions 234 and 235 of the lower hinge region alone and in combination with substitutions in the CH2 domain at P329 have also been reported in the art (see e.g., U.S. Pat. No. 5,624,821 (SB2 Inc.) disclosing inter alia L234A and L235E; U.S. Pat. Nos. 8,969,526 and 10,093,714 (Roche) disclosing P329G or R and L234A/L235A; U.S. Pat. No. 6,528,624 (Genentech) disclosing P329A; Idusogie et al., J. Immunol. (2000) 164:4178-4184, disclosing P329A prevents C1q interaction). While P329A was reported to have no effect on Fc binding to FcγRI and FcγRII receptors and only a very small decrease in Fc binding to the FcγRIIIA receptor (see Table 1 and Table 2 of U.S. Pat. No. 6,528,624), the same substitution was conversely shown, by a different group, to reduce binding to FcγRI, FcγRII, and FcγRIIIA receptors (Shields, et al., J. Biol. Chem. 276(9):6591-6604 (2001)). P329G was reported to inhibit Fc binding to FcγRs and, thus, was selected for combination with L234A and L235A to provide a variant Fc with reduced effector function, whereas P329A was once again shown to exhibit wildtype-like (or near) binding to FcγRs and, accordingly, deemed an unfavorable mutation for reducing effector function (see U.S. Pat. No. 8,969,526). Therefore, while much effort has gone in to engineering a “silent” Fc region, there is still a need for antibodies with strongly decreased effector function.

### SUMMARY OF THE INVENTION

The invention relates to polypeptides comprising a variant Fc region (Fc variant) with reduced affinity for human FcγRs and C1q. It has been found that the Fc variants described herein provide several improvements over the wildtype IgG Fc region, e.g., higher titer, as well as previously reported Fc variants, e.g., reduced C1q binding and/or reduced FcγRI binding.

In particular, the polypeptides comprise a Fc variant containing an amino acid substitution at each of Pro329, Leu234, and Leu235 (EU numbering) and, as a result, exhibit (i) a reduced affinity to human FcγRIIIA and FcγRIIA and FcγRI as compared to a polypeptide comprising the wildtype IgG Fc region, and (ii) reduced C1q binding as compared to a polypeptide comprising the wildtype IgG Fc region.

A polypeptide is provided that comprises a Fc variant of a parent Fc, which variant contains amino acid substitutions L234A, L235A, and P329A (LALA-PA). The LALA-PA polypeptide may exhibit reduced affinity to each of FcγRI, FcγRIIA, FcγRIIIA, and C1q as compared to a polypeptide comprising the wildtype human Fc region.

In some embodiments, the LALA-PA polypeptide is an antibody or Fc fusion protein.

In some embodiments, the parent Fc polypeptide is a wildtype human IgG1 Fc region or a wildtype human IgG4 Fc region.

In some embodiments, the LALA-PA polypeptide maintains FcRn binding comparable to the parent Fc polypeptide.

In some embodiments, the affinity of the LALA-PA polypeptide for each of FcγRI, FcγRIIA, and FcγRIIIA is reduced by greater than 95% as compared to the polypeptide comprising the wildtype human Fc region. In some embodiments, the affinity of the LALA-PA polypeptide for C1q is reduced by greater than 80% as compared to the polypeptide comprising the wildtype human Fc region. In some embodiments, the titer of the peptide is at least four-fold greater as compared to the polypeptide comprising the wildtype human Fc region.

Also provided is a polypeptide comprising a Fc variant of a parent Fc, which variant contains amino acid substitutions L234A, L235E, and P329G (LALE-PG).

In some embodiments, the LALE-PG polypeptide may exhibit reduced affinity to each of FcγRI, FcγRIIA, FcγRIIIA, and C1q as compared to a polypeptide comprising the wildtype human Fc region.

In some embodiments, the polypeptide is an antibody or Fc fusion protein.

In some embodiments, the parent Fc polypeptide is a wild-type human IgG1 Fc region.

In some embodiments, the parent Fc polypeptide is a wild-type human IgG4 Fc region.

In some embodiments, the polypeptide maintains FcRn binding comparable to the parent Fc polypeptide.

In some embodiments, the affinity of the polypeptide for each of FcγRI, FcγRIIA, and FcγRIIIA is reduced by greater than 95% as compared to the polypeptide comprising the wildtype human Fc region.

In some embodiments, the affinity of the polypeptide for C1q is reduced by greater than 75% as compared to the polypeptide comprising the wild-type human Fc region.

In some embodiments, the titer of the polypeptide is at least four-fold greater as compared to the polypeptide comprising the wild-type human Fc region.

In some embodiments, the polypeptide lacks any of the following Fc mutations: E233P, AG236 (deletion of residue 236), D265A, N297A, N297D, and P331S.

In some embodiments, the polypeptide does not comprise any other Fc mutations which reduce binding to any of FcγRI, FcγRIIA, FcγRIIIA, and C1q.

In some embodiments, the polypeptide does not comprise any Fc mutations other than substitutions L234A, L235E, and P329G, wherein numbering is according to the EU index.

In some embodiments, a multispecific antibody comprising a Fc variant according to any of the foregoing is provided.

In some embodiments a human or humanized antibody comprising a Fc variant according to any of the foregoing is provided.

In some embodiments nucleic acids encoding any of the foregoing Fc variant polypeptides are provided.

In some embodiments expression vectors comprising nucleic acids encoding any of the foregoing Fc variant polypeptides are provided.

In some embodiments isolated or recombinant cells are provided which comprise a nucleic acid encoding any of the foregoing Fc variant polypeptides or an expression vector containing same.

In some embodiments methods of producing a Fc variant polypeptide according to any of the foregoing are provided comprising culturing a cell comprising a nucleic acid encoding any of the foregoing Fc variant polypeptides under conditions that result in expression of the polypeptide and optionally isolating the polypeptide from the cell or cell culture containing same.

In some embodiments pharmaceutical compositions that comprise the LALA-PA polypeptide or the LALE-PG polypeptide are provided.

### BRIEF DESCRIPTION OF THE DRAWING

FIG. 1 shows CD3 antibody variant binding to FcγR on THP-1 cells and C1q Dyna Beads. WT: wild-type IgG1 anti-CD3 antibody; LALA: WT engineered to contain L234A and L235A; LALA-PA: WT engineered to contain L234A and L235A and P329A; LALA-PG: WT engineered to contain L234A and L235A and P329G. MFI=mean fluorescence intensity (measure of antibody binding).

FIG. 2 shows CD3 antibody variant binding to FcγR on THP-1 cells and C1q Dyna Beads. WT: wild-type IgG1 anti-CD3 antibody; LALE: WT engineered to contain L234A and L235E; LALE-PA: WT engineered to contain L234A and L235E and P329A; LALE-PG: WT engineered to contain L234A and L235E and P329G. MFI=mean fluorescence intensity (measure of antibody binding).

FIG. 3 shows CD3 antibody variant binding to FcRn, FcγRI, FcγRIIA, FcγRIIIA, and C1q measured using Biacore. WT: wild-type IgG1 anti-CD3 antibody; LALA: WT engineered to contain L234A and L235A; LALA-PA: WT engineered to contain L234A and L235A and P329A; LALA-PG: WT engineered to contain L234A and L235A and P329G. RU=resonance units (measure of complex formation).

FIG. 4 shows CD3 antibody variants binding to FcRn, FcγRI, FcγRIIA, FcγRIIIA, and C1q measured using Biacore. WT: wild-type IgG1 anti-CD3 antibody; LALE: WT engineered to contain L234A and L235E; LALE-PA: WT engineered to contain L234A and L235E and P329A; LALE-PG: WT engineered to contain L234A and L235E and P329G. RU=resonance (measure of complex formation).

### DETAILED DESCRIPTION OF THE INVENTION

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

It is understood that aspects and embodiments of the disclosure described herein include “comprising,” “consisting,” and “consisting essentially of” aspects and embodiments.

The disclosure relates to polypeptides comprising a variant Fc domain (variant Fc), which has several advantages compared to a parent Fc domain. A “variant Fc” refers to an Fc region that has been modified relative to a parent Fc region. A variant Fc may comprise a human Fc region sequence (e.g., a human IgG1, IgG2, IgG3 or IgG4 Fc region) comprising a modification at one or more amino acid positions in the Fc region. Such modifications include substitution of a wild-type amino acid at a particular position, i.e., 234, 235 and 329, or replacement of the amino acid at each of these positions to the amino acid residues specified herein, i.e., 234A+235A+329A or 234A+235E+329G, in the Fc region. The “Fc region” is a C-terminal region of an immunoglobulin heavy chain that spans the domains CH2, CH3, and a portion of the hinge region. A human IgG heavy chain Fc region can extend from Cys226, or from Pro230, to the carboxyl-terminus of the heavy chain. However, the C-terminal lysine (Lys447) of the Fc region may or may not be present. “Lower hinge” of the Fc region refers to amino acid residues found from about position 230 to about position 237. A “parent Fc” refers to a starting or nonvariant or wild-type Fc domain, which may be prepared using techniques available in the art for generating antibodies or other polypeptides (such as immunoadhesins) comprising an Fc region.

The Fc variants described herein comprise amino acid substitutions (relative to the parent amino acid sequence) to decrease or minimize effector function. “Effector function” refers to biological activities attributable to the Fc region of an antibody, which varies by antibody isotype. Exemplary effector functions include: C1q binding and complement dependent cytotoxicity (CDC); Fc receptor binding (including Fc gamma receptors and FcRn); antibody-dependent cell-mediated cytotoxicity (ADCC); phagocytosis; down regulation of cell surface receptors (e.g., B cell receptor); and B cell activation.

“Fc gamma receptor” or “FcγR” refers to any member of the family of proteins that bind the IgG antibody Fc region and is encoded by an FcγR gene. This family includes FcγRI (CD64), including isoforms FcγRIA, FcγRIB, and FcγRIC; FcγRII (CD32), including isoforms FcγRIIA (including allotypes H131 and R131), FcγRIIB (including FcγRIM-1 and FcγRIIB-2), and FcγRIIc; and FcγRIII (CD16), including isoforms FcγRIIIA (including allotypes V158 and F158) and FcγRIIIb (including allotypes FcγRIM-NA1 and FcγRIIB-NA2) (Jefferis, et al., Immunol Lett 82 (2002) 57-65, herein incorporated by reference in its entirety), as well as any undiscovered human FcγRs or FcγR isoforms or allotypes. An FcγR may be from any organism, including, but not limited to, humans, mice, rats, rabbits, and monkeys.

“FcRn” or “neonatal Fc Receptor” refers to a protein that binds the IgG antibody Fc region and is encoded at least in part by an FcRn gene. The FcRn may be from any organism, including, but not limited to, humans, mice, rats, rabbits, and monkeys. A functional FcRn protein comprises two polypeptides—a heavy chain and a light chain. The light chain is beta-2-microglobulin and the heavy chain is encoded by the FcRn gene. Unless otherwise noted, FcRn or an FcRn protein refers to the complex of FcRn heavy chain with beta-2-microglobulin.

The Fc variants of the present invention may possess some but not all effector functions, such that a peptide comprising such Fc variant is a desirable therapeutic candidate for applications in which certain effector functions (such as ADCC and CDC) are unnecessary or deleterious.

“Antibody dependent cell-mediated cytotoxicity” (ADCC) refers to a process by which nonspecific cytotoxic cells that express FcRs recognize bound antibody on a target cell and subsequently cause lysis of the target cell (Ravetch, et al., Annu Rev Immunol 19 (2001) 275-290). “Antibody dependent cell-mediated phagocytosis” (ADCP) refers to a process where nonspecific cytotoxic cells that express FcRs recognize bound antibody on a target cell and subsequently cause phagocytosis of the target cell. For the IgG class of antibodies, ADCC and ADCP are governed by engagement of the Fc region with a family of receptors referred to as Fcγ receptors (FcγRs). In humans, this family includes FcγRI (CD64); FcγRII (CD32), including isoforms FcγRIIA, FcγRIIB, and FcγRIIC; and FcγRIII (CD16), including isoforms FcγRIIIA and FcγRIIIB (Raghavan, and Bjorkman, Annu. Rev. Cell Dev. Biol. 12 (1996) 181-220; Abes, et al., Expert Reviews 5(6), (2009) 735-747). ADCC activity may be determined using any number of methods known in the art including flow-cytometry-based assays (e.g., Wilkinson et al, J. Immunol. Methods, 256(1-2), (2001) 183-191), an ADCC-reporter gene assay based on key attributes of peripheral blood mononuclear cell (PBMC)-based assays (e.g., Parekh, et al., MABS 4(3), 2012 310-318), ⁵¹Cr release assays (e.g., Perussia et al., Methods Mol. Biol. 121 (2000) 179-192), and/or cell-based assays employing Natural Killer cell lines engineered to express a high affinity variant of FcγRIIIa (V158) (e.g., Thomann et al., PLoS One (2015) 10:e0134949).

“Complement dependent cytotoxicity” (CDC) refers to the mechanism by which antibody-coated target cells recruit and activate components of the complement cascade, leading to the formation of a Membrane Attack Complex (MAC) on the cell surface and subsequent cell lysis. The CDC mechanism is mediated by Fc binding to the complement protein C1q. C1q is a polypeptide that includes a binding site for the Fc region of an immunoglobulin. C1q, together with two serine proteases, C1r and C1s, forms the complex C1, the first component of the complement dependent cytotoxicity (CDC) pathway. C1q is capable of binding six antibodies, although binding to two IgGs is sufficient to activate the complement cascade. CDC activity may be determined using any number of methods known in the art including cell viability assays using redox dyes like Alamar blue (e.g., Gazzano-Santoro et al., J. Immunol. Methods, 202(2), (1997) 163-171), viability assays using fluorescent labels like carboxyfluorescein diacetate succinymyl ester and 70 amino-actinomycin D (e.g., Sawada et al., Clin Cancer Res, 17(5), (2011) 1024-1032), and ⁵¹Cr release assays (e.g., Kato et al., Oncotarget, 6(34), (2015) 36003-36018).

In vitro and/or in vivo cytotoxicity assays can be conducted to confirm reduction/depletion of CDC and/or ADCC activities. For example, Fc receptor (FcR) binding assays can be conducted to ensure that an antibody lacks FcγR binding (hence likely lacking ADCC activity) but retains FcRn binding ability. The primary cells for mediating ADCC (e.g. NK cells), express FcγRIII only, whereas monocytes express FcγRI, FcγRII and FcγRIII. FcR expression on hematopoietic cells is taught in Ravetch et al., Ann. Rev. Immunol. 9:457-492 (1991). Non-limiting examples of in vitro assays to assess ADCC activity of a molecule of interest are described in: U.S. Pat. No. 5,500,362 (see, e.g. Hellstrom, I. et al. Proc. Nat'l Acad. Sci. USA 83:7059-7063 (1986)) and Hellstrom, et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (Cell Technology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Also, ADCC activity may be assessed in vivo, e.g., in an animal model such as that disclosed in Clynes et al. Proc. Nat'l Acad. Sci. USA 95:652-656 (1998). C1_{q} binding assays may also be carried out to confirm that an antibody is unable to bind C1q and hence lacks CDC activity. See, e.g., WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, et al., Blood 103:2738-2743 (2004)). FcRn binding and in vivo clearance/half-life determinations can also be performed using methods known in the art (see, e.g., Petkova, et al. Int'l. Immunol 18(12):1759-1769 (2006)).

The Fc variant comprising L234A; L235A; P329A (LALA-PA) and the Fc variant comprising L234A; L235E; P329G (LALE-PG), both of which are provided herein, display significantly reduced binding to FcRs and C1_{q} and, accordingly, may provide reduced effector functions associated with and/or mediated by FcγRs and C1q, e.g., ADCC and CDC, respectively.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of a native IgG molecule.

Each set of substitutions was tested for their ability to reduce binding to FcRs and not FcRn. In some embodiments, retaining an antibody's ability to bind FcRn is important for maintaining half-life. Testing for reduced FcR binding included measuring THP-1 cell binding as well as binding affinities for FcγRI and FcγRIIIA Additional developability tests were conducted (e.g., PSR, pH stress, Tm, etc.) to verify that these substitutions did not have developability issues. Lower hinge substitutions LALA and LALE were found to significantly reduce FcR binding and both displayed good developability profiles.

Results showed that LALA+P329A (“LALA-PA”) and LALE+P329G (“LALE-PG”) substitutions significantly reduced FcR and C1q binding while maintaining FcRn binding and maintaining good developability profiles. In some embodiments, the LALA-PA variant or the LALE-PG variant reduce affinity for each of FcγRI, FcγRIIA, and FcγRIIIA by greater than 95% as compared to a polypeptide comprising a wild-type human Fc region. In some embodiments, the LALA-PA or LALE-PG substitutions reduce affinity for each of FcγRI, FcγRIIA, and FcγRIIIA by at least about 100%, at least about 99%, at least about 98%, at least about 97%, at least about 96%, at least about 95%, at least about 94%, at least about 93%, at least about 92%, at least about 91%, at least about 90%, and/or all percentages in between, compared to a wildtype Fc region. In some embodiments, the LALA-PA or LALE-PG substitutions reduce affinity for C1q by at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, and/or all percentages in between, compared to a wildtype Fc region. In one embodiment, LALE-PG reduces affinity for C1q by about 75%. In another embodiment, LALA-PA reduces affinity for C1q by about 80%. These substituted combinations provide superior reduction of Fc function compared to previously known amino acid substitution combinations. For example, the LALE-PG substitutions provide a larger reduction in binding to THP-1 compared to LALA-PG (Roche, U.S. Pat. No. 8,969,526), and the LALA-PA substitutions provided a larger reduction in binding to C1q compared to LALA-PG (see Table 1).

In some embodiments, a polypeptide comprising the Fc variant is an antibody or an Fc fusion protein. “Fc fusion protein” refers to a polypeptide comprising an Fc region fused to a non-immunoglobulin polypeptide (e.g., ligand-binding domain, ligand, enzyme, or peptide epitope).

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity).

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)₂; diabodies; linear antibodies; single-chain antibody molecules (e.g. scFv or V_{HH} or V_{H} or V_{L} domains only); and multispecific antibodies formed from antibody fragments. In some embodiments, the antigen-binding fragments of the antibodies described herein are scFvs.

As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

A “multi specific antibody” refers to an antibody comprising at least two different antigen binding domains that recognize and specifically bind to at least two different antigens. A “bispecific antibody” is a type of multispecific antibody and refers to an antibody comprising two different antigen binding domains that recognize and specifically bind to at least two different antigens.

A “different antigen” may refer to different and/or distinct proteins, polypeptides, or molecules; as well as different and/or distinct epitopes, which epitopes may be contained within one protein, polypeptide, or other molecule.

The term “epitope” refers to an antigenic determinant that interacts with a specific antigen binding site in the variable region of an antibody molecule known as a paratope. A single antigen may have more than one epitope. Thus, different antibodies may bind to different areas of an antigen and may have different biological effects. The term “epitope” also refers to a site of an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

In some instances, an antibody comprises four polypeptide chains: two heavy (H) chains and two light (L) chains interconnected by disulfide bonds. Each heavy chain comprises a heavy chain variable region (“V_{H}”) and a heavy chain constant region (“CH”), which comprises domains CH1, CH2 and CH3. Each light chain comprises a light chain variable region (“V_{L}”) and a light chain constant region (“CL”). The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences or may be naturally or artificially modified. An amino acid consensus sequence may be defined based on a side-by-side analysis of two or more CDRs. The CDRs in a heavy chain are designated “CDRH1”, “CDRH2”, and “CDRH3”, respectively, and the CDRs in a light chain are designated “CDRL1”, “CDRL2”, and “CDRL3”. In other instances, an antibody may comprise multimers thereof (e.g., IgM) or antigen-binding fragments thereof.

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

As described throughout, the antibodies and/or antigen-binding fragments thereof as provided herein possess favorable developability and are, thus, relatively developable.

The term “developable” refers to the extent to which one or more polypeptides in a plurality of polypeptides possess desirable characteristics, such as, e.g., desirable expression, for example, in mammalian cells; solubility; viscosity; aggregation; chemical and/or physical stability; desirable shelf-life; melting temperature; pharmacokinetic profiles; circulation half-life; and clearance characteristics. Such characteristics may serve as indicia, independently, as combinations of sub-sets of such indicia, or in totality, for the likelihood that such one or more polypeptides may be successfully developed as a therapeutic candidate, and ultimately an approved drug. Accordingly, as understood in the art, generally, polypeptides with desirable developability characteristics possess, e.g., relatively high solubility, relatively low viscosity, relatively low propensity for aggregation, relatively high chemical stability, relatively high physical stability, relatively long shelf life, relatively high melting temperature, relatively long circulation half-life, relatively long clearance time, and the like. Polypeptides with undesirable developability characteristics possess, e.g., relatively low solubility, relatively high viscosity, relatively high propensity for aggregation, relatively poor chemical stability, relatively poor physical stability, relatively short shelf life, relatively low melting temperature, relatively short circulation half-life, relatively short clearance time, and the like.

Methods and assays that may be employed to ascertain the degree to which polypeptides, such the antibodies and/or antigen-binding fragments thereof as described herein, possess desirable developability characteristics are available in the art, and include, for example; PSR assays (WO 2014/179363 and Xu et al., Protein Eng Des Sol, Vol. 26, pages 663-670 (2013)); SMP and SCP assays and the like; cross interaction chromatography (CIC); self-interaction chromatography (SIC); dynamic light scattering; size exclusion chromatography (SEC), dynamic light scattering (DLS) spectroscopy; photon correlation spectroscopy; quasi-elastic light scattering, circular dichroism (CD), viscosity measurements; whole cell binding; tissue micro array methodologies; BVP ELISA assays; AC-SINS assays (Liu et al; mAbs, Vol. 6, pages 483-492 (2014); differential scanning calorimetry; and the like (see, e.g., He et al., J. Pharm. Sci., Vol. 100(4), pp. 1330-1340 (2011); Wagner et al., Pharm Dev Technol, 18(4):963-970 (2013), (posted online 2012; hyper-text transfer protocol: informahealthcare.com/doi/abs/10.3109/10837450.2011.649851); Hotzel et al., mAbs, Vol. 4(6):753-760 (2012); Cheng et al., J. Pharm. Sci., Vol. 101(5), pp. 1701-1720 (2012); Banks et al., J. Pharm. Sci., Vol. 101(8), pp. 2720-2732 (2012); Lie et al., J. Pharm. Sci., Vol. 94(9), pp. 1928-1948 (2005); and Payne et al., Biopolymers, Vol. 85(5), pp. 527-533 (2006)).

In some embodiments, antibodies that are identified as possessing decreased developability are so detected by virtue of their interaction with a polyspecificity reagent (“PSR”) and, as such, are referred to as “polyspecific” polypeptides. Such polyspecific antibodies may be referred to as relatively “undevelopable” or relatively “non-developable”.

In other embodiments, the antibodies and/or antigen-binding fragments thereof as described herein display an increased titer relative to wild-type, i.e., improved expressability. In some embodiments, the titer of the LALA-PA variant or the LALE-PG variant is at least about two-fold, at least about three-fold, at least about four-fold, at least about five-fold, at least about six-fold, greater relative to wildtype.

Examples are provided below to illustrate the present invention. These examples are not meant to constrain the present invention to any particular application or theory of operation. For all positions discussed in the present invention, numbering is according to the EU index as in Kabat (Kabat et al., 1991, Sequences of Proteins of Immunological Interest, 5^{th} Ed., United States Public Health Service, National Institutes of Health, Bethesda). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

### EXAMPLES

### Example 1: Antibodies

For the following experiments, antibodies against CD3 (described in WO/2018/208864) were used.

All variants described herein—LALA, LALA-PA, LALA-PG, LALE, LALE-PA, LALE-PG variants of the CD3 antibody (numbering according to EU nomenclature)—were generated using PCR based mutagenesis of the parental antibody (WT; ADI-26906). IgGs were expressed in HEK cells and purified using protein A chromatography.

### Example 2: THP-1 Cell Binding and C1q Binding to Antibodies

LALA, LALA-PA, LALA-PG, LALE, LALE-PA, LALE-PG variants were tested for the ability to bind THP-1 and C1q.

100 nM of each Fc variant antibody was pre-complexed with 10 nM biotinylated CD-3 peptide at room temperature for 20 minutes. Pre-complexed antibodies were added directly to THP-1 cells (200,000 cells per well) and incubated for 30 minutes on ice. Cells were washed three times with PBS and then incubated with 100 μl of goat anti-human IgG PE antibody (Southern Biotech) at a 1:200 dilution for 20 minutes at room temperature. After two washes in PBS, cells were resuspended in 100 μl of PBS and analyzed on a FACS Canto (BD Biosciences) for PE intensity on live cells. Mean fluorescence intensity (MFI) of each sample was recorded.

Biotinylated CD3-peptide was incubated with streptavidin (SA)-coupled Dyna Beads (Thermofisher) for 15 minutes at room temperature. Beads were washed in PBS to remove unbound CD3 peptide and transferred to a 96-well plate at a density of 50 μg of beads per well in 48 wells. The beads were then incubated with saturating amounts (100 μl of 100 nM) of wild-type or Fc variant antibodies for 15 minutes. A 96 well plate magnet was used to wash the samples three times in PBS. Samples were next incubated with C1q alone or with 100 μl of human complement (C3) for 25 minutes and then washed three times with PBS. Samples were incubated with 100 μl of goat anti-human IgG PE antibody (Southern Biotech at a 1:200 dilution for 20 minutes at room temperature. Samples were then collected on a FACS Canto (BD Biosciences) and the mean fluorescence intensity (MFI) of each sample was recorded.

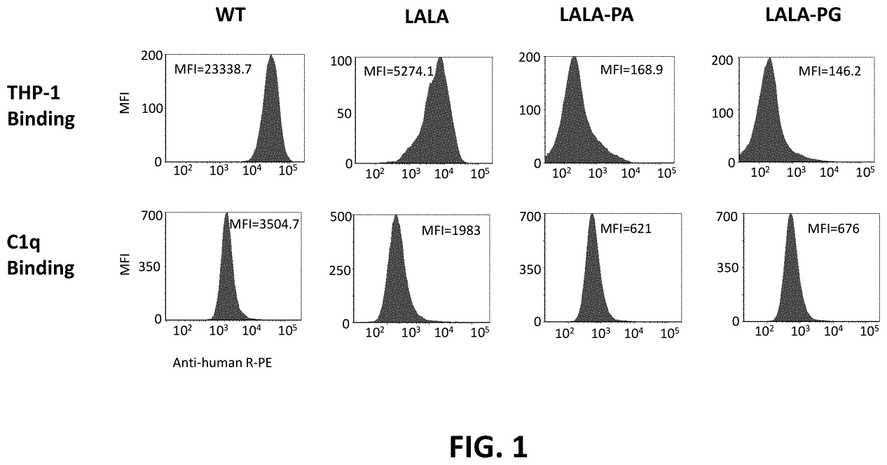

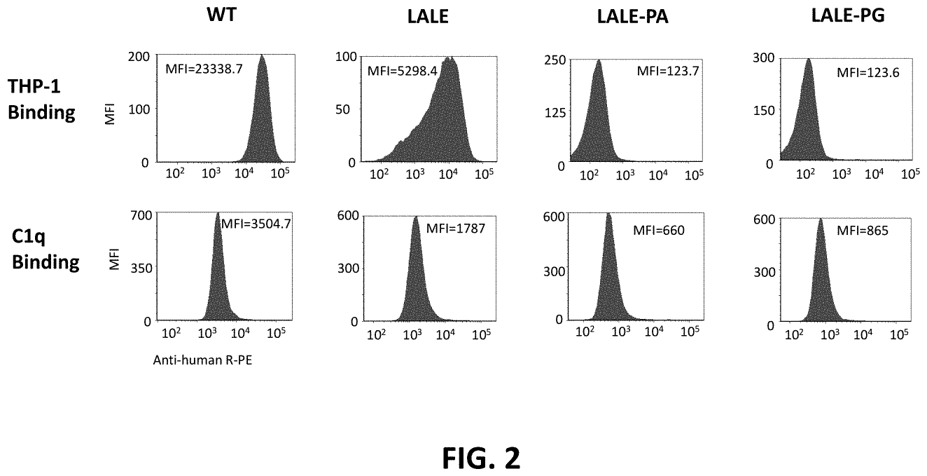

LALA-PA and LALE-PG displayed significantly reduced binding to THP-1 cells and C1q as compared to wild-type (WT) (THP-1 binding: WT 23338.7 MFI; LALA-PA 168.9 MFI; LALE-PG: 123.6 MFI; C1q binding: WT 3505 MFI; LALA-PA 621 MFI; LALE-PG: 865 MFI). FIG. 1 shows exemplary THP-1 and C1q binding data for LALA-PA compared to LALA-PG, LALA, and WT, and FIG. 2 shows exemplary binding data for LALE-PG compared to LALE-PA, LALE, and WT. A summary of THP-1 cell binding data and C1q binding data is provided in Table 1. Of note, (i) LALE-PG provided a larger reduction in binding to FcγRs compared to LALA-PG (LALE-PG: 123.6 MFI vs LALA-PG: 146.2 MFI), and (ii) LALA-PA provided a larger reduction in binding to C1q compared to LALA-PG (LALA-PA: 621 MFI vs LALA-PG: 676 MFI).

TABLE 1
Binding data for lower hinge plus CH2 substitutions

| | THP-1 Binding | Clq Binding |
|---|---|---|
| Substitutions | (MFI) | (MFI) |
| WT | 23338.7 | 3505 |
| LALA | 5274.1 | 1983 |
| LALA-PA | 168.9 | 621 |
| LALA-PG | 146.2 | 676 |
| LALE | 5298.4 | 1787 |
| LALE-PA | 123.7 | 660 |
| LALE-PG | 123.6 | 865 |

### Example 3: FcγR Binding by Antibodies

Binding characteristics of antibodies with Fc variant domains were also assessed by surface plasmon resonance (SPR) using a Biacore 8K instrument (GE Healthcare BioSciences, Marlborough, Mass.).

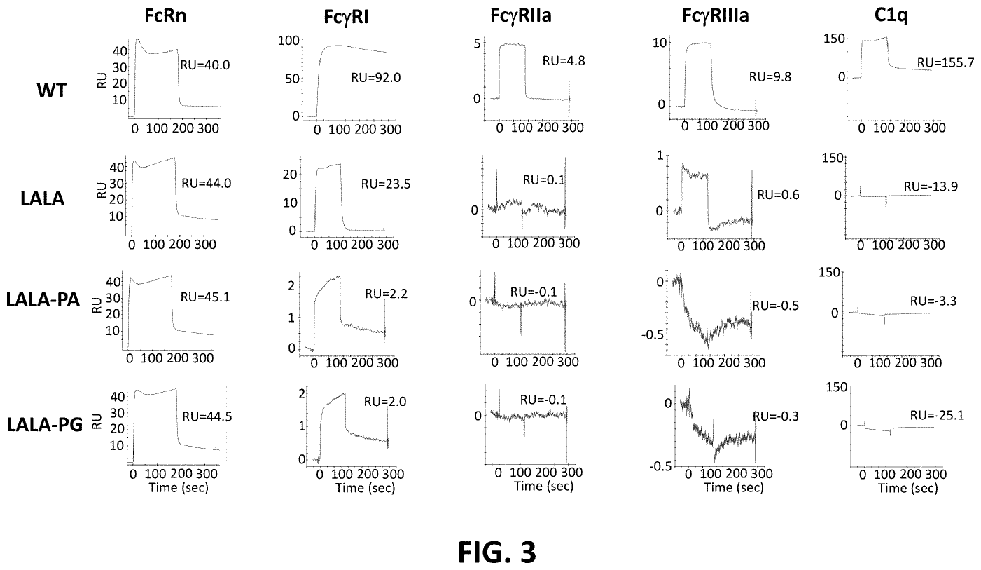

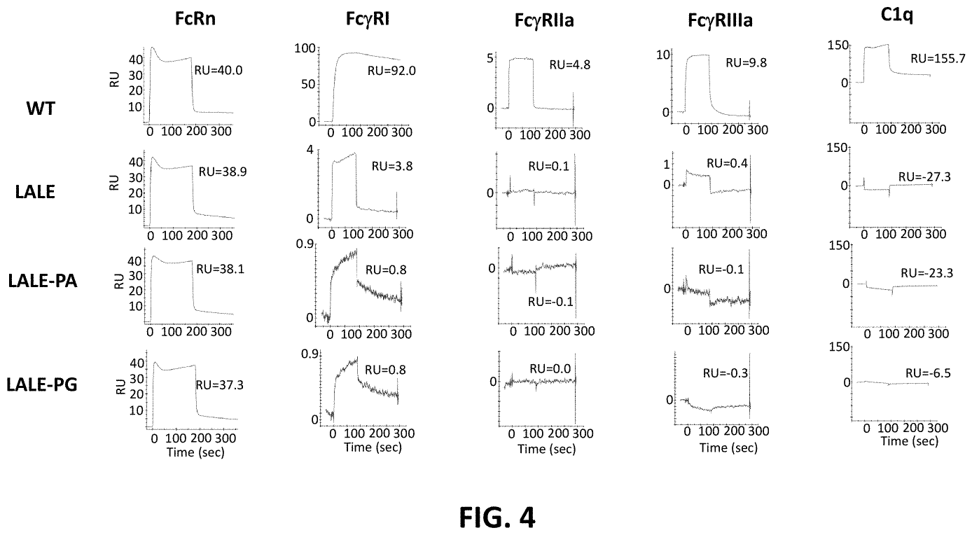

SPR-based binding response measurements were performed by flowing solutions of potential binding partners over a sensor chip surface affixed with the antibodies of interest. For binding to analytes FcγRI, FcγRIIA, FcγRIIIA, FcRn and C1q, a streptavidin (SA) sensor chip immobilized (35 RU) with a biotinylated-CD3 peptide was used to capture (450 RU) the IgGs of interest. Upon IgG capture, the interaction between each individual analyte and the IgG was measured by flowing the analyte solutions (100 nM for FcγRI; 1 μM for FcγRIIA, FcγRIIIA, FcRn and C1q) over flow cells. Dissociation of the bound analytes were measured by flowing instrument running buffer over flow cells for 3-5 minutes. Binding to FcγRI, FcγRIIA, FcγRIIIA, and C1q were performed at pH 7.4, while binding to FcRn was performed at pH 6.0. The resulting sensorgrams were double reference subtracted using the Biacore Evaluation software version 1.0. Relative binding responses (Late Association Response measured in resonance units (RU)) near the end of the analyte binding step were recorded for each interaction. Table 2 summarizes the binding responses for wild-type and each Fc variant. FIG. 3 shows exemplary binding data for LALA-PA compared to LALA-PG, LALA, and WT. FIG. 4 shows exemplary binding data for LALE-PG compared to LALE-PA, LALE, and WT. Results show that the P329A substitution in combination with LALA reduced binding to FcγRI, FcγRIIA, FcγRIIIA, and C1q by greater than 95% compared to WT (FcγRI: 2.2 RU vs. 92 RU (97.6% reduction); FcγRIIA: −0.1 RU vs. 4.8 RU (100% reduction); FcγRIIIA: −0.5 RU vs. 9.8 RU (100% reduction); C1q: (−3.3 RU vs. 155.7 RU (100% reduction)). Further, the LALA-PA variant reduced binding of some FcγRs compared to LALA alone (e.g., FcγRI: 2.2 RU vs 23.5 RU). Results also show that the P329G substitution in combination with LALE significantly reduced binding of FcγRs and C1q compared to WT (FcγRI: 0.8 RU vs. 92 RU (99.1% reduction); FcγRIIA: 0 RU vs. 4.8 RU (100% reduction); FcγRIIIA: −0.3 RU vs. 9.8 RU (100% reduction); C1q: −6.5 RU vs. 155.7 RU (100% reduction)). The LALE-PG variant also significantly reduced binding of some FcγRs compared to LALE alone (e.g., FcγRI: 0.8 RU vs. 3.8 RU).

TABLE 2
Fc variant binding to FcRn, FcγRI, FcγRIIA, FcγRIIIA, and C1q

| Substitutions | FcRn | FcγRI | FcγRIIA | FcγRIIIA | C1q |
|---|---|---|---|---|---|
| WT | 40.0 | 92.0 | 4.8 | 9.8 | 155.7 |
| LALA | 44.0 | 23.5 | 0.1 | 0.6 | −13.9 |
| LALE | 38.9 | 3.8 | 0.1 | 0.4 | −27.3 |
| LALA-PA | 45.1 | 2.2 | −0.1 | −0.5 | −3.3 |
| LALA-PG | 44.5 | 2.0 | −0.1 | −0.3 | −25.1 |
| LALE-PA | 38.1 | 0.8 | −0.1 | −0.1 | −23.3 |
| LALE-PG | 37.3 | 0.8 | 0.0 | −0.3 | −6.5 |

### Example 4: Developability Assays

LALA, LALA-PA, LALA-PG, LALE, LALE-PA, LALE-PG variants were tested using the following assays to determine developability characteristics: polyreactivity (PSR), retention time (HIC), self-association (AC-SINS), aggregation (ProA SEC), pH stress, titer, melting temperature (Tm), and CD3 affinity.

PSR Preparation: Polyspecific reactivity reagent (PSR) was prepared as described in, e.g., WO 2014/179363 and Xu et al., Protein Eng Des Sol, 26: 663-670 (2013). In brief, 2.5 liters CHO—S cells were used as starting material. The cells were pelleted at 2,400×g for 5 minutes in 500 mL centrifuge bottles filled to 400 mL. Cell pellets were combined and then resuspended in 25 mL Buffer B and pelleted at 2,400×g for 3 minutes. The buffer was decanted and the wash repeated one time. Cell pellets were resuspend in 3× the pellet volume of Buffer B containing 1× protease inhibitors (Roche, Complete, EDTA-free) using a polytron homogenizer with the cells maintained on ice. The homogenate was then centrifuged at 2,400×g for 5 minutes and the supernatant retained and pelleted one additional time (2,400×g/5 min) to ensure the removal of unbroken cells, cell debris and nuclei; the resultant supernatant was the total protein preparation. The supernatant was then transferred into two Nalgene Oak Ridge 45 mL centrifuge tubes and pelleted at 40,000×g for 40 min at 4° C. The supernatants containing the Separated Cytosolic Proteins (SCPs) were then transferred into clean Oak Ridge tubes, and centrifuged at 40,000×g one more time. In parallel, the pellets containing the membrane fraction (EMF) were retained and centrifuged at 40,000 for 20 minutes to remove residual supernatant. The EMF pellets were then rinsed with Buffer B. 8 mL Buffer B was then added to the membrane pellets to dislodge the pellets and transfer into a Dounce Homogenizer. After the pellets were homogenized, they were transferred to a 50 mL conical tube and represented the final EMF preparation.

One billion mammalian cells (e.g. CHO, HEK293, Sf9) at approximately 106-107 cells/mL were transferred from tissue culture environment into 4×250 mL conical tubes and pelleted at 550×g for 3 minutes. All subsequent steps were performed at 4° C. or on ice with ice-cold buffers. Cells were washed with 100 mL of PBSF (1×PBS+1 mg/mL BSA) and combined into one conical tube. After removing the supernatant, the cell pellet was then re-suspended in 30 mL Buffer B (50 mM HEPES, 0.15 M NaCl, 2 mM CaCl₂, 5 mM KCl, 5 mM MgCl₂, 10% Glycerol, pH 7.2) and pelleted at 550×g for 3 minutes. Buffer B supernatant was decanted and cells re-suspended in 3× pellet volume of Buffer B plus 2.5× protease inhibitor (Roche, cOmplete, EDTA-free). Protease inhibitors in Buffer B were included here and subsequently. Cells were homogenized four times for 30 second pulses (Polyton homogenizer, PT1200E) and the membrane fraction was pelleted at 40,000×g for 1 hour at 4° C. The pellet was rinsed with 1 mL Buffer B; the supernatant was retained and represented the soluble cytoplasmic polyspecific reagent (SCP). The pellet was transferred into a Dounce homogenizer with 3 mL of Buffer B and re-suspended by moving the pestle slowly up and down for 30-35 strokes. The enriched membrane fraction (EMF) was moved into a new collection tube, and the pestle was rinsed to collect all potential protein. The protein concentration of the purified EMF was determined using a Dc-protein assay kit (BioRad). To solubilize the EMF, the EMF was transferred into Solubilization Buffer (50 mM HEPES, 0.15 M NaCl, 2 mM CaCl₂, 5 mM KCl, 5 mM MgCl₂, 1% n-Dodecyl-b-D-Maltopyranoside (DDM), 1× protease inhibitor, pH 7.2) to a final concentration of 1 mg/mL. The mixture was rotated overnight at 4° C., followed by centrifugation in a 50 mL Oak Ridge tube (Fisher Scientific, 050529-ID) at 40,000×g for 1 hour. The supernatant, which contained the soluble membrane proteins (SMPs), was collected and the protein yield was quantified as described above.

In order to prepare biotinylated EMF, HS-LC-Biotin stock solution was prepared according to manufacturer's protocol (Pierce, Thermo Fisher). In brief, 20 μl of biotin reagent was added for every 1 mg of EMF sample and incubated at 4° C. for 3 hours with gentle agitation. The volume was adjusted to 25 mL with Buffer B and transferred to an Oak Ridge centrifuge tube. The biotinylated EMF (b-EMF) was pelleted at 40,000×g for 1 hour, and rinsed two times with 3 mL of Buffer C (Buffer B minus the glycerol) without disturbing the pellet. The residual solution was removed. The pellet was re-suspended with a Dounce homogenizer in 3 mL of Buffer C as described previously. The re-suspended pellet contained biotinylated EMF (b-EMF) and was solubilized as described above to prepare b-SMPs.

PSR Binding Analyses: Assays were performed generally as described in, e.g., Xu et al. To characterize the PSR profile of monoclonal antibodies presented on yeast, two million IgG-presenting yeast were transferred into a 96-well assay plate and pellet at 3000×g for 3 minutes to remove supernatant. The pellet was re-suspended in 50 μL of a freshly prepared 1:10 dilution of stock b-PSRs and incubated on ice for 20 minutes. The cells were washed twice with 200 μL of cold PBSF and the pellet was re-suspended in 50 μL of secondary labeling mix (Extravidin-R-PE, anti-human LC-FITC, and propidium iodide). The mix was incubated on ice for 20 minutes followed by two washes with 200 μL ice-cold PB SF. The cells were re-suspended in 100 μL of ice-cold PBSF and the plate was run on a FACSCanto (BD Biosciences) using HTS sample injector. Flow cytometry data was analyzed for mean fluorescence intensity in the R-PE channel and normalized to proper controls in order to assess non-specific binding. Numerous methods for presentation or display of antibodies or antibody fragments on the surface of yeast have been described previously, all of which are consistent with this protocol (Blaise et al., Gene, 342(2): 211-218, 2004, Boder and Wittrup, Nat. Biotechnol., 15, 553-557, 1997, Kuroda and Ueda, Biotechnology Letters, 33:1-9, 2011, Orcutt and Wittrup, Antibody Engineering, “Yeast Display and Selections”, pp. 207-223, 2010, Rakestraw et al., Protein Eng. Des. Sel., 24, 525-530, 2011, Sazinsky et al., Proc. Natl Acad. Sci. U.S.A., 105, 20167-20172, 2008, Tasumi et al., Proc Natl Acad. Sci U.S.A., 106:12891-6, 2009, Vasquez et al., Adimab Inc., “Rationally designed, synthetic antibody libraries and uses therefor”, WO/2009/036379, 2009.

HIC Retention Time Analyses: IgG1 samples were buffer exchanged into 1 M ammonium sulfate and 0.1 M sodium phosphate at pH 6.5 using a Zeba 40 kDa 0.5 mL spin column (Thermo Pierce, cat #87766). A salt gradient was established on a Dionex ProPac HIC-10 column from 1.8 M ammonium sulfate, 0.1 M sodium phosphate at pH 6.5 to the same condition without ammonium sulfate. The gradient ran for 17 minute at a flow rate of 0.75 mL/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate.

AC-SINS (self-interaction): Antibody samples were incubated with citrate-stabilized gold nanoparticles (20 nm) coated with polyclonal goat anti-human Fc antibodies. Particle plasma wavelength was then measured. Self-associative antibodies captured on the gold nanoparticle surface caused particle precipitation and thus a red shift in the plasma wavelength.

ProA Size Exclusion Chromatography (SEC) Analyses: A TSKgel SuperSW mAb HTP column (22855) was used for fast SEC analysis of yeast produced mAbs at 0.4 mL/min with a cycle time of 6 min/run. 200 mM Sodium Phosphate and 250 mM Sodium Chloride was used as the mobile phase.

pH Stress Analyses: 1 mg/mL mAb or Fab solution was incubated at pH 3.5 for 1 hour. Samples were then neutralized and analyzed for aggregates by SEC as described above.

Dynamic Scanning Fluorimetry Analyses (Tm): 10 uL of 20× Sypro Orange was added to 20 μL of 0.2-1 mg/mL mAb or Fab solution. An RT-PCR instrument (BioRad CFX96 RT PCR) was used to ramp the sample plate temperature from 40° to 95° C. at 0.5° C. increments, with a 2 minute equilibration at each temperature. The negative of the first derivative for the raw data was used to extract Tm.

HEK Titer: Wild-type or Fc variant antibodies were expressed in HEK293 cells grown in shake flasks. After six days of growth, the cell culture supernatant was harvested by centrifugation and passed over Protein A agarose (Mab Select Sure (GE Healthcare Life Sciences)). Bound antibodies were washed with PBS and eluted with buffer consisting of 200 mM acetic acid and 50 mM NaCl at pH3.5 into 1/10th volume 2 M HEPES, pH 9.0. Antibody titer was calculated by multiplying the purified antibody concentration by its final volume and dividing by the volume that was transfected.

ForteBio KD measurements (Biolayer interferometry; BLI): CD3 affinity was confirmed using ForteBio measurements performed generally as previously described (Estep, P., et al., High throughput solution-based measurement of antibody-antigen affinity and epitope binning. MAbs, 2013. 5(2): p. 270-8.). Briefly, ForteBio affinity measurements were performed by loading IgGs online onto AHQ sensors. Sensors were equilibrated off-line in assay buffer for 30 minutes and then monitored on-line for 60 seconds for baseline establishment. Sensors with loaded IgGs were exposed to 100 nM antigen for 5 minutes were then transferred to assay buffer for 5 minutes for off-rate measurement. Kinetics was analyzed using the 1:1 binding model.

LALA-PA and LALE-PG variants maintained good developability profiles as measured by melting temperature (Tm), self-association (AC-SINS), aggregation (ProA SEC), pH stress, titer, PSR, HIC, and CD3 affinity. These results are summarized in Table 3. LALA-PA and LALE-PG variants showed improved developability compared to wild-type in some assays such as titer. LALA-PA and LALE-PG had titers of 246 mg/L and 265 mg/L, respectively, as compared to wild-type titer of 54 mg/L.

TABLE 3
Developability data for Fc variants

| | | | | pH | | | | |
|---|---|---|---|---|---|---|---|---|
| | Fc | AC- | ProA | Stress | | | | CD3 |
| Substi- | Tm | SINS | SEC | SEC | Titer | | HIC | Binding |
| tutions | (° C.) | (nm) | (%) | (%) | (mg/L) | PSR | (min) | (K_{D} (M)) |
| WT | 67.5 | 8.1 | 97.6 | 0.7 | 54 | 0.33 | 8.5 | 3.4E−09 |
| LALA | 66.5 | 9.1 | 93.8 | −0.2 | 224 | 0.37 | 8.4 | 3.7E−09 |
| LALA-PA | 67.0 | 10.8 | 94.9 | −0.4 | 246 | 0.38 | 8.4 | 3.9E−09 |
| LALA-PG | 66.5 | 12.8 | 98.0 | 1.0 | 222 | 0.38 | 8.4 | 3.7E−09 |
| LALE | 66.5 | 11.2 | 96.6 | 0.5 | 316 | 0.37 | 8.4 | 3.7E−09 |
| LALE-PA | 66.0 | 11.3 | 97.4 | 0.7 | 257 | 0.36 | 8.4 | 3.7E−09 |
| LALE-PG | 65.0 | 10.5 | 96.5 | 0.8 | 265 | 0.35 | 8.4 | 3.6E−09 |

### Example 5: ADCC Activity Assay

ADCC activity of the Fc variants described herein can be assessed using the ADCC Reporter Bioassay (Promega Corp). Briefly, target cells (e.g., Raji or WIL2-S; 4×10⁴ cells in assay medium) are added to the wells of a 96-well assay plate. Serial dilutions of wild-type or Fc variant antibodies are added to the target cells and the mixture is incubated for 30 minutes at 37° C. with 5% CO₂ to allow opsonization. Engineered Jurkat effector cells (that express FcγRIIIa) are then added to each well, and the plate is incubated for 4 hours. After incubation, the plate is cooled to room temperature, and the Bio-Glo Reagent (Promega) is added. The plate is incubated again and then is read using a Synergy Multi-Mode Microplate Reader (BioTek) in luminescence mode.

### Example 6: Complement Activity Assay

CDC activity of the Fc variants described herein can be assessed using a luminescence-based assay using WIL2-S cells as target cells (Promega). Wild-type or Fc variant antibodies are serially diluted in assay medium (RPMI 1640 medium with 1% FBS) and distributed into a 96-well opaque-walled microtiter plate. The plate is incubated with 55 CO₂ for 2 hours at 37° C. after WIL2-S cells (5×10⁴ cells/well) and normal human serum complement are added. After incubation, a CellTiter-Glo reagent that assays for ATP in metabolically active cells is added and the plate is incubated for 10 minutes at room temperature with constant shaking. Cell lysis is quantified by measuring intensity of luminescence with a plate reader.

What is claimed is:

1. A polypeptide comprising a Fc variant of a parent Fc polypeptide, said Fc variant comprising amino acid substitutions L234A, L235E, and P329G, wherein numbering is according to the EU index.

2. The polypeptide of claim 1, wherein the polypeptide exhibits reduced affinity to each of FcγRI, FcγRIIA, FcγRIIIA, and C1q as compared to a polypeptide comprising the wild-type human Fc region.

3. The polypeptide of claim 1, wherein the polypeptide is an antibody or Fc fusion protein.

4. The polypeptide of any one of claim 1, wherein the parent Fc polypeptide is a wild-type human IgG1 Fc region.

5. The polypeptide of any one of claim 1, wherein the parent Fc polypeptide is a wild-type human IgG4 Fc region.

6. The polypeptide of any one of claim 1, wherein the polypeptide maintains FcRn binding comparable to the parent Fc polypeptide.

7. The polypeptide of any one of claim 1, wherein the affinity of the polypeptide for each of FcγRI, FcγRIIA, and FcγRIIIA is reduced by greater than 95% as compared to the polypeptide comprising the wildtype human Fc region.

8. The polypeptide of any one of claim 1, wherein the affinity of the polypeptide for C1q is reduced by greater than 75% as compared to the polypeptide comprising the wild-type human Fc region.

9. The polypeptide of any one of claim 1, wherein the titer of the polypeptide is at least four-fold greater as compared to the polypeptide comprising the wild-type human Fc region.

10. The polypeptide of any one of claim 1, wherein the polypeptide lacks any of the following Fc mutations: E233P, AG236 (deletion of residue 236), D265A, N297A, N297D, and P331S.

11. The polypeptide of any one of claim 1, wherein the polypeptide does not comprise any other Fc mutations which reduce binding to any of FcγRT, FcγRIIA, FcγRIIIA, and C1q.

12. The polypeptide of any one of claim 1, wherein the polypeptide does not comprise any Fc mutations other than substitutions L234A, L235E, and P329G, wherein numbering is according to the EU index.

13. A multispecific antibody comprising a Fc variant according to any one of claim 1.

14. A human or humanized antibody comprising a Fc variant according to any one of claim 1.

15. A pharmaceutical composition comprising a polypeptide according to any one of claim 1.

16. A nucleic acid encoding a polypeptide according to any one of claim 1.

17. An expression vector comprising a nucleic acid according to claim 16.

18. An isolated or recombinant cell which comprises a nucleic acid or expression vector according to claim 16.

19. A method of producing a Fc variant polypeptide according to claim 1, comprising culturing a cell comprising a nucleic acid encoding said Fc variant polypeptide under conditions that result in the expression of the Fc variant polypeptide and optionally isolating the Fc variant polypeptide from the cell or cell culture containing same.
